(19) United States
(12) Patent Application Publication
(10) Pub. No.: US 2021/0236778 A1
(43) Pub. Date: Aug. 5, 2021

(54) APPARATUS AND METHOD FOR DEPLOYING MEDICAL DEVICE USING STEERABLE GUIDEWIRE

(71) Applicants: BOARD OF REGENTS OF THE UNIVERSITY OF TEXAS SYSTEM, Austin, TX (US); XCATH, INC., Houston, TX (US)

(72) Inventors: Daniel H. KIM, Houston, TX (US); Dong Suk SHIN, Houston, TX (US)

(21) Appl. No.: 17/154,719

(22) Filed: Jan. 21, 2021

Related U.S. Application Data
(60) Provisional application No. 62/965,400, filed on Jan. 24, 2020.

(51) Int. Cl. A61M 25/09; A61B 34/30; A61B 17/221; A61B 17/12; A61M 25/10; A61K 51/12

(52) U.S. Cl. CPC A61M 25/09; A61B 34/30; A61B 17/221; A61B 2017/22079; A61M 25/104; A61B 17/1214; A61K 51/1255; A61B 17/12113

(57) ABSTRACT

Provided herein as systems, apparatus, and methods for the effective and minimally invasive deployment of medical devices or therapeutic agents using a steerable guidewire/catheter assembly. The steerable guidewire and/or catheter has a bendable distal portion comprising an ionic electroactive polymer actuator, which is allowed to deform, bend or expand from its original shape in at least one dimension.

### CROSS-REFERENCE TO RELATED APPLICATIONS

This application claims benefit of U.S. provisional patent application Ser. No. 62/965,400, filed Jan. 24, 2020, which is herein incorporated by reference.

### BACKGROUND

### Field

The present specification relates to surgical apparatuses, systems and procedures using steerable guidewires. More specifically, the present specification is related to surgical apparatuses, systems and procedures for controlling, navigating and deploying medical devices to a targeted site.

### Description of the Related Art

Introduction catheters or guidewires are known as a deploying mechanism to deliver an element, such as a stent, a balloon, or other device, to a desired location in a body lumen. In these applications, a guidewire is introduced into the body lumen, and an end thereof is steered to a desired location in the body lumen by visually tracking the location of a radiopaque marker on the end thereof which is visualized radiologically by a surgeon. This steering can include moving the orientation of the distal tip of the guidewire with respect to the remainder thereof, to steer the tip into tortuous portions of the lumen or into branch lumens, etc. Once the distal tip of the guidewire is properly positioned in the body, a catheter, which may include a deployable element such as the stent or balloon thereon or therein, is advanced over the wire to position the distal end thereof in a desired location (such as a blood vessel in the brain) within the patients' body.

One issue with such catheter/guidewire deployment systems is the limited ability to conform the distal end of the guidewire to follow tortuous lumen geometries, as well as to guide the distal end of the guidewire into an intersecting lumen or branch lumen from the lumen the distal end of the guidewire is currently in. To guide the distal end of the guidewire into a branch lumen, the distal end of the guidewire must be controllably moved from alignment with the lumen in which it reached the branching lumen location to an alignment where further movement of the guidewire inwardly of the body will cause the guidewire to enter and follow the branch lumen. In some cases, the branch lumen, a location of which is the target destination of the distal end of the guidewire, intersects the lumen in which the distal end is present at a large angle, for example greater than forty-five, degrees, and in some cases greater than ninety degrees.

As can be seen, there is a need for apparatuses, systems, and methods for the safe, reliable, and minimally invasive deployment of medical devices or therapeutic agents to the target sites.

### SUMMARY

The present invention provides systems, apparatus, and methods for the effective and minimally invasive deployment of medical devices or therapeutic agents. In an embodiment the invention provides a minimally invasive method to deploy a medical device or therapeutic agent using a guidewire, catheter, or guidewire and catheter assembly. The guidewire, catheter, or guidewire and catheter assembly is steerable to deform, bend or expand (swell) from its original shape in at least one dimension.

According to the present invention, a method for deploying a medical device to a target site of a patient is provided, comprising: a) introducing a steerable guidewire to a body lumen of a patient, the steerable guidewire having a bendable distal portion comprising an ionic electroactive polymer actuator; b) steering the bendable distal portion of the guidewire to a target site of the body lumen with a guidewire controller; c) introducing and advancing a flexible, elongated catheter having a distal end over the guidewire to position the distal end in the target site; and d) deploying a deployable element through the catheter to the target site.

In some embodiments, the guidewire of b) can be removed when the bendable distal portion is positioned.

In some embodiments, the guidewire controller of b) can be a hand-held controller.

In some embodiments, the both movement of the guidewire and catheter can be controlled by a robotic controller. For example, in some other embodiments, the robotic controller may comprise: a catheter driver configured to advance and retract a catheter having, a hollow interior, along a catheter advance and retract path extending therein; a guidewire driver configured to advance and retract a guidewire along a guidewire advance and retract path extending therein; wherein each of the catheter advance and retract path and the guidewire advance and retract path extend between pairs of rollers in the respective catheter and roller drivers, and the paths are parallel to each another.

In some embodiments, the deployable element may include a thrombectomy device, for example, but not limited to, a mechanical retriever or aspiration assembly for removing clots (i.e. occlusions).

In some embodiments, the deployable element may include a detachable coil for the treatment of aneurysm.

In some embodiments, the deployable element may include a balloon or a stent for angioplasty.

In some embodiments, the deployable element may include a therapeutic agent for example, but not limited to a plurality of microsphere beads for embolization or radioactive seeds for tumor treatment

These and other features, aspects, and advantages of the present invention will become better understood with reference to the following drawings, description, and claims.

### BRIEF DESCRIPTION OF THE DRAWINGS

FIG. 1A is a schematic view of a portion of the human vasculature according to one embodiment to one embodiment of the present invention. FIG. 1B is an enlarged section view of a brain vessel of FIG. 1A. FIG. 1C is a schematic view of endovascular navigation in the human vasculature of FIG. 1A with a steerable guidewire and a catheter according to one embodiment of the present invention.

FIG. 2A is a schematic view of a portion of a steerable guidewire according to one embodiment of the present invention, showing a section of the interior arrangement of an intermediate portion, an electrical connection portion 14 and controllably bendable portion 16.

FIG. 2B is an enlarged view of FIG. 2A illustrates the controllably bendable portion and a portion of the adjacent hypotube section.

FIG. 3 is a schematic view of the manual controller of the steerable guidewire according to one embodiment of the present invention.

FIG. 4A is an isometric view of the robotic controller assembly of the steerable guidewire and the catheter shown in FIG. 1.

FIG. 4B is an isometric view of the distal end portions of the catheter and guidewire of FIG. 4.

FIG. 5 is a plan view of the robotic controller assembly, shown in FIG. 4A.

FIGS. 6A and B are schematic views illustrating a deployable element being deployed through the steerable guidewire/catheter system to remove the occlusion according to one embodiment of the present invention. FIG. 6A illustrates catheter of FIG. 1 being advanced through the vasculature to reach an occlusion within from a patient's blood vessel. FIG. 6B illustrates a thrombectomy device is deployed adjacent to the occlusion.

FIGS. 7A to C are schematic views illustrating another deployable element being deployed through the steerable guidewire/catheter system to perform angioplasty according to one embodiment of the present invention. FIG. 7A illustrates a balloon catheter being advanced to reach plaques of the blood vessel. FIG. 7B illustrates an inflatable balloon at the catheter distal end being inflated to expand a stent thereon. FIG. 7C illustrates the stent being left within the blood vessel when the balloon is deflated and retracted from the catheter.

FIGS. 8A and B are schematic views illustrating another deployable element being deployed through the steerable guidewire/catheter system to perform embolization according to one embodiment of the present invention. FIG. 8A illustrates a catheter being advanced to reach a desired portion of the blood vessel supplying blood flow to a tumor or abnormal area of tissues. FIG. 8B illustrates a plurality of degradable microspheres being deployed and accumulated to a portion of the blood vessel.

FIGS. 9A to C are schematic views illustrating another deployable element being deployed through the steerable guidewire/catheter system to perform radiation therapy according to one embodiment of the present invention. FIG. 9A illustrates a catheter being advanced through the vasculature 2 to reach a desired portion of the blood vessel 20 adjacent to a tumor area. FIG. 9B illustrates that when the catheter distal end is properly positioned, as shown in FIG. 9B, to deploy a plurality of radioactive seeds into or around the tumor area. FIG. 9C illustrates different doses of radiation being given off from radioactive seeds to the tumor area.

### DETAILED DESCRIPTION

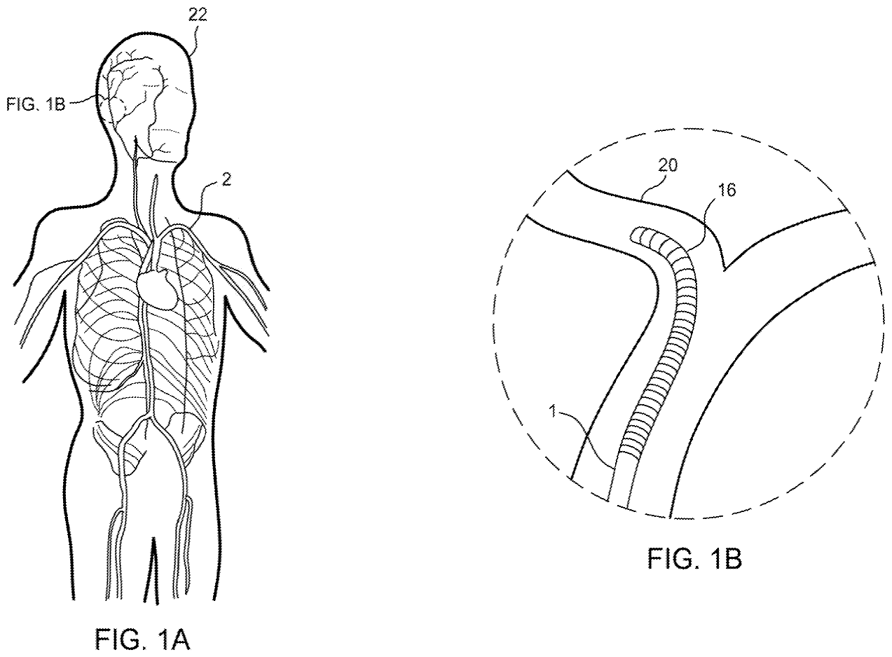

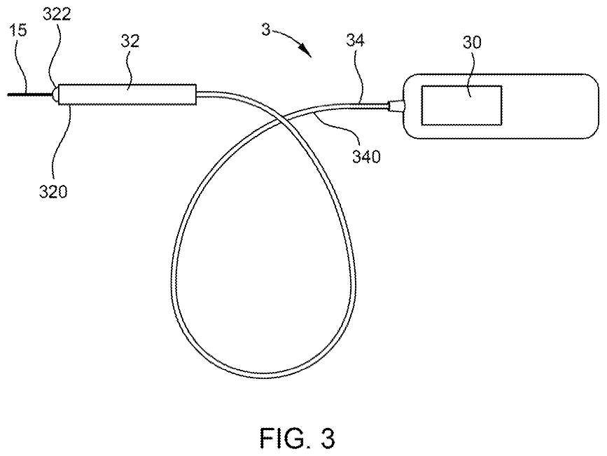

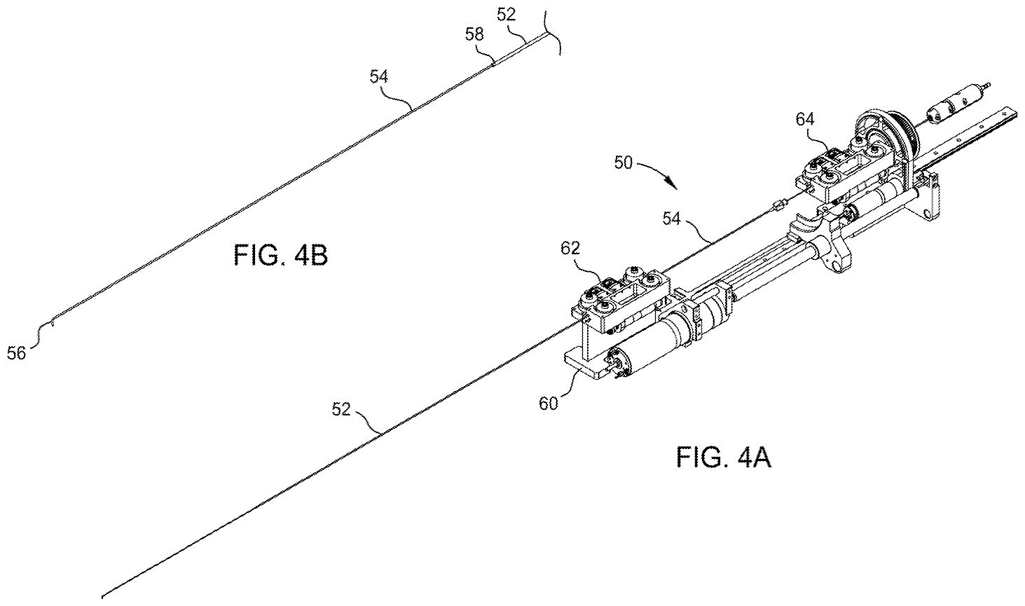

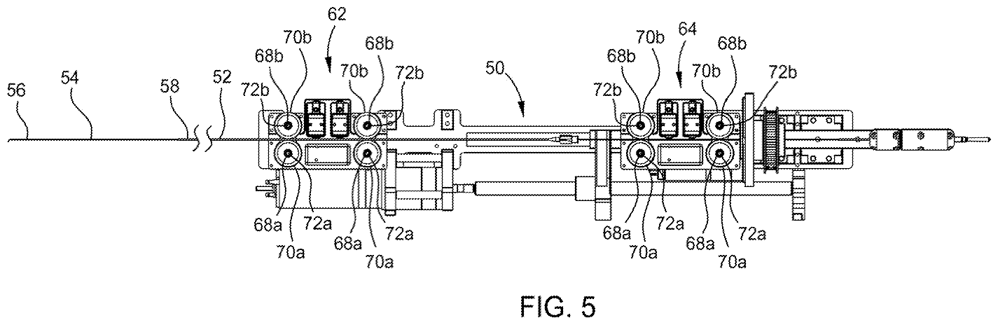

FIG. 1A is a schematic view of a portion of the human vasculature according to one embodiment to one embodiment of the present invention. FIG. 1B is an enlarged sectional view of a brain blood vessel of FIG. 1A cross A. FIG. 1C is a schematic view of endovascular navigation in the human vasculature of FIG. 1A with a steerable guidewire and a catheter according to one embodiment of the present invention. The human vasculature 2, as shown in FIG. 1A, includes various tiny tortuous blood vessels and complicated arrangements thereof in organs, which makes it difficult to deliver medical devices or therapeutic agents to a desired location therewithin. To preciously deploy such elements, a steerable guidewire 1 having a controllably bendable portion 16 as a “steerable tip” is introduced into the vasculature 2, and steered to navigate within the blood vessel 20 in a patient's brain 22 to a desired location in response to control signals sent from, and motion created by, a manual controller 3 (see e.g. FIG. 3) or a robotic controller 50 (see, e.g. FIG. 4) operated by an operator (not shown). A microcatheter or catheter 52 is further guided by, i.e., over, the steerable guidewire 1 to advance its distal end 58 toward, or retract its distal end 58 away from, the desired location.

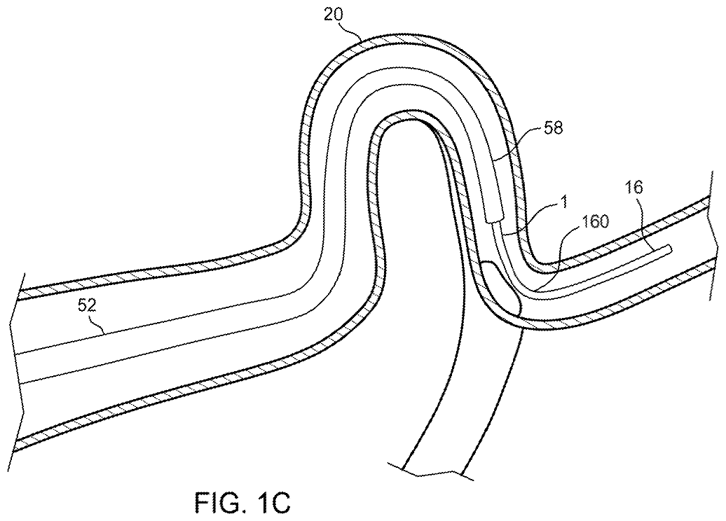

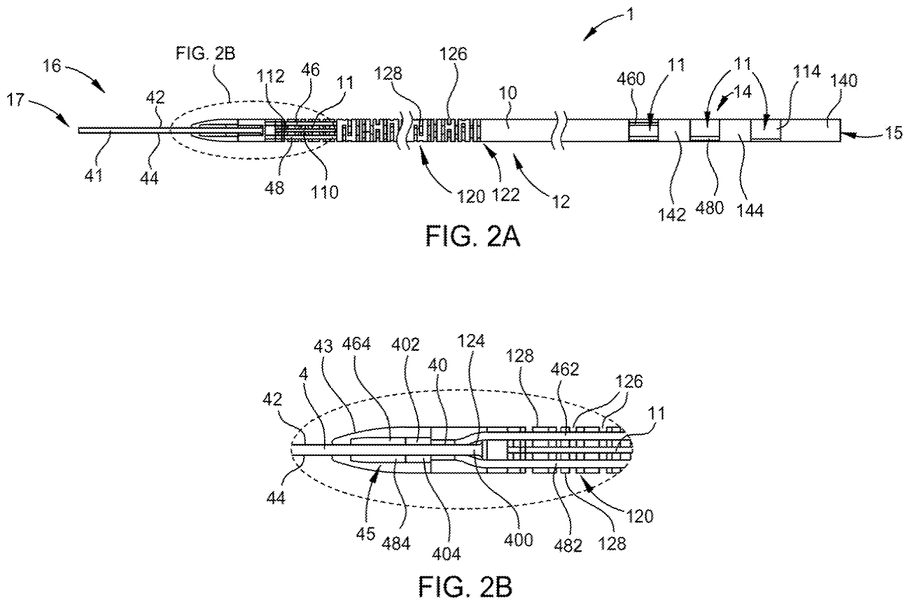

FIG. 2A is a schematic view of a steerable guidewire according to one embodiment of the present invention, such as that disclosed in No. WO 2019/212863 and No. WO 2019/027826, which are incorporated herein by reference in their entireties, except for any definitions, subject matter disclaimers or disavowals, and except to the extent that the incorporated material is inconsistent with the express disclosure herein, in which case the language in this disclosure controls. The guidewire 1 includes a hollow, tubular shaft 10 and an electrically conductive tapered core 11 therein. The shaft 10 defines an intermediate portion 12 thereof, an electrical connection portion 14 formed adjacent to the proximal end 15 thereof, and a controllably bendable portion 16 formed at the distal end 17 of the guidewire 1. Here, FIG. 2A is further partially shown in section to show the interior arrangement of these portions. The tapered core 11 extends inwardly within shaft 10 from the proximal end 15 thereof to the distal end 17 thereof and has a tapered end 110 connecting to the controllably bendable portion 16 at the distal end 17 thereof. The intermediate portion 12 further comprises a hypotube section 120 between a first end 122 of the intermediate portion 12 and the controllably bendable portion 16. Here, the guidewire 1 is shown extended in a generally straight line path, and, the bendable portion 16 in its free state, i.e. without an electrical bias applied thereacross.

As shown in FIG. 2B, in one aspect, the controllably bendable portion 16 is configured from an electroactive polymer portion 4 sandwiched between electrodes 42, 44 directly formed on, or bonded to, opposed sides of the electroactive polymer portion 4. In one embodiment, the electroactive polymer portion 4 is an ionic electroactive polymer actuator comprising a polymer electrolyte member 41 made of PVDF-HFP that is impregnated with EMITF (as an electrolyte). Alternately, other embodiments of a polymer electrolyte member comprise a perfluorinated ionomer such as Aciplex™ (available from Asahi Kasei Chemical Corp. of Tokyo, Japan), Flemion® (available from AGC Chemical Americas, Inc. of Exton, Pa., USA), fumapem® F-series (available from Fumatech BWT GmbH, Bietigheim-Bissingen, Federal Republic. Electrodes 42, 44 can be made by pressing and adhering a carbon layer (not shown) respectively on both faces (i.e., surface on opposed sides thereof) of the polymer electrolyte member 41, followed by overlying metal electrodes 42, 44 over the carbon layer, such that a highly electrically conductive path is formed to distribute electricity over the length and width of both faces of the polymer electrolyte member 41, and thereby maintain a uniform voltage potential across the respective electrodes 42, 44. For example, the electrodes 42, 44 may be formed of a sputtered or vapor deposited layer of gold, silver, palladium or platinum, wherein each electrode has the same, or nearly the same, thickness. The carbon layer may comprise carbon-based materials such as carbide-derived carbon, carbon nanotube, graphene, a composite of carbide-derived carbon and polymer electrolyte member, and a composite of carbon nanotube and polymer electrolyte member. Alternatively, electrodes 42, 44 can be made formed of a shape memory material, for example a NiTi alloy or a NiTi based alloy, formed on the controllably bendable portion 16 by sputtering, vapor deposition or other deposition or adhering methods as discussed in No. WO 2019/027826. When applying a bias or potential across electrodes 42, 44, cations within the polymer electrolyte member 41 will migrate towards an anodically energized electrode, and away from a cathodically energized electrode, while remaining within the matrix of the polymer electrolyte member 41. This causes a portion of the polymer electrolyte member 41 adjacent to an anodically energized electrode to swell and a portion of the polymer electrolyte member 41 adjacent to a cathodically energized electrode to contract, thereby causing electroactive polymer portion 4 to bend.

To connect the electroactive polymer portion 4 to the intermediate portion 12, the hypotube section 120 here is a thin walled conductive sleeve, for example, a bio-compatible stainless steel tube with a receiving slot 124 having a slot height or width slightly larger than the thickness of the electroactive polymer portion 4. By configuring the width of the receiving slot 124 slightly larger than the thickness of the electroactive polymer portion 4, the first side 40 of the electroactive polymer portion 4 can be spaced from, and thus electrically isolated from, the inner wall of the receiving slot 124. The hypotube section 120 further includes a plurality of cross cut slots 126 cut inwardly thereof from opposed circumferential sides thereof, to leave a pair of opposed webs 128 extending circumferentially between some of the pairs of slots.

The electrical connection portion 14 includes a detection terminal 140 at the proximal end 15 electrically connecting to detection electrodes (not shown) of the manual controller 3 (see FIG. 3) for detecting the engagement of the guidewire 1 therewith, a first terminal 142 connecting to a proximal end 460 of a first wire 46, and a second terminal 144 connecting to a proximal end 480 of a second wire 48, while the distal ends 462, 482 of wires 46, 48 are connected to the electrodes 42, 44 respectively on the controllably bendable portion 16 with a conductive adhesive (such as a gold paste) to form terminals 464, 484, thereby providing an electric flow connection, i.e., an electrical circuit, between the wires 46, 48 and the terminals 464, 484 and from the manual controller 3. The first wire 46 having a conductive core and a surrounding insulation, and the second wire 48 having a conductive core and a surrounding insulation, extend within the hypotube section 120. Here, the conductive portions of the wires 46, 48 are configured of a base metal such as stainless steel and are covered with a thin layer of gold, but other conductive material coverings, such as silver, copper, cobalt or rhenium or ruthenium may be used as the conductor. Since the electrical connection of the power source to the opposed electrodes 42, 44 of the electroactive polymer portion 4 is provided through dedicated wires 46, 48 surrounded by an insulation, the distal end 112 of the tapered core 11 and the hypotube section 120 need not be electrically isolated from one another. However, the proximal end 114 of the tapered end is still insulated to connect the proximal terminal 140 and the control and power connector 32.

In addition, the proximal end 400 of the electroactive polymer portion 4 is further covered with a layer of encapsulant 43, 45 respectively, composed of, for example, a silicone adhesive, to encapsulate the radiopaque marker plates (not shown), for example, composed of a platinum iridium alloy and disposed slightly inwardly of the proximal end 400 of the electroactive polymer portion 4 on both sides 402, 404 thereof. The encapsulant 43, 45, and the adjacent portion of the hypotube section 120 are dipped into a silicone dispersion to be coated therewith, and then the hypotube section 120, and the dip coated electroactive polymer and encapsulant 43, 45 extending therefrom, are vapor coated with a coating of, for example, parylene. The vapor coated electroactive polymer portion 4, the encapsulant 43, 45, and the adjacent portion of the hypotube section 120 are again dipped into a silicone dispersion to be coated therewith, the hypotube section 120, and the dip coated electroactive polymer and encapsulant 43, 45 are again coated with a vapor coating of, for example, parylene, and then the parylene coating is covered with a hydrophilic coating to complete the assembly of the guidewire 1.

FIG. 3 is a schematic view of the manual controller of the steerable guidewire according to one embodiment of the present invention, such as that disclosed in No. WO 2019/212863, which are incorporated herein by reference in their entireties, except for any definitions, subject matter disclaimers or disavowals, and except to the extent that the incorporated material is inconsistent with the express disclosure herein, in which case the language in this disclosure controls. The manual controller 3 is configured as a battery-powered, disposable design, including a control and power module 30, a control and power connector 32, an electrical lead portion 34 having a flexible tubular protective covering 340 thereover and which extends from the control and power module 30 and terminates at the connector 32. The proximal end 15 of the guidewire 1 is receivable in an opening in the end wall of the distal end 320 of the connector 32. The connector 32 includes a warning light 322 at its distal end 320. When the detection terminal 140 of the guidewire 1 electrically connects to the detection electrodes (not shown) within the connector 32, the warning light 322 is green, indicating the guidewire 1 is engaged to the manual controller 3. And, when the warning light 322 is red, it indicates the guidewire 1 is not engaged to the manual controller 3 or the detection terminal 140 thereof is not in electrical connection with the detection electrodes (not shown) of the connector 32. The manual controller 3 delivers electrical current to the controllable bendable portion 16 with the operation of two-way directional control buttons on the control and power module 30, and the profile of the controllable bendable portion 16 can be thus steered to form, for example, the desired bend 160 (or bends) of the controllably bendable portion 16 within the blood vessels as shown in FIG. 1C.

Alternatively, both the movement of the steerable guidewire and catheter can be controlled by a robotic controller. FIG. 4 is a schematic view of the robotic controller according to one embodiment of the present invention, such as that disclosed in No. WO 2019/133438, which are incorporated herein by reference in their entireties, except for any definitions, subject matter disclaimers or disavowals, and except to the extent that the incorporated material is inconsistent with the express disclosure herein, in which case the language in this disclosure controls. In FIG. 4, the robotic controller 50, having the catheter 52, and the steerable guidewire 54 discussed above extending therefrom (FIG. 4A), is shown isometrically. In this construct of the robotic controller, the robotic controller 50 includes a catheter driver 62 and a guidewire driver 64, wherein the catheter driver 62 is fixedly connected to a base 60 of the robotic controller fixed to the guide tray (not shown), and the guidewire driver 64 is slideably connected to the base 60. In this construct, each of the catheter driver 62 and the guidewire driver 64 are configured to enable fine movement control of the positioning of the guidewire 54 and catheter 52 with respect to the base 60, and the guidewire 54 is additionally moveably relative to the catheter 52 by moving the guidewire driver 64 slideably with respect to the base 60. Thus, if each of the catheter driver 62 and guidewire driver 64 are held stationary with respect to each other while sliding with respect to the base 60, they each simultaneously move the catheter 52 and guidewire 54 toward, or away from, the opening on guide tray (not shown) to which the base 60 is attached, the catheter 52 and guidewire 54 will not move with respect to each other despite moving with respect to the opening on the guide tray ea (not shown), and thus through an incision in the patient and thus within the a patient's body. Alternatively, where the catheter driver 62 moves the catheter 52 with respect thereto, the guidewire driver 64 may be itself moved, with the guidewire 54 remaining stationary with respect thereto, to effect simultaneous movement of the catheter 52 and guidewire 54 with respect to the opening, but not with respect to each other. In either case, as the guidewire is inserted into tortuous anatomy, the bendable distal portion 56 of the guidewire 54 may also be controllably advanced away from, or retracted toward or even into, the distal end 58 of the catheter 52, by operation of the catheter driver 62 and the guidewire driver 54 with a controller. Additionally, by moving the guidewire 54 with respect to the catheter, such as by moving the guidewire driver 64 with respect to the catheter driver 62 while holding the guidewire stationary with respect to the guidewire driver 64, and holding the catheter 52 stationary with respect to the catheter drive 62, the portion of the guidewire 54 extending outwardly of the distal end of the catheter 64 can be modified. This can also be accomplished by moving the guidewire 54 within the guidewire driver 64 at a desired rate while the catheter is held stationary by the catheter driver 62 or moved at a different rate by the catheter driver 62 than the rate of movement of the guidewire. Alternatively this can also be accomplished by moving the catheter 52c within the catheter driver 62 at a desired rate while the guidewire 54 is held stationary by the guidewire driver 64 or moved at a different rate by the guidewire driver 64 than the rate of movement of the guidewire. Movement of the guide wire driver 64 and catheter driver 62 with respect to each other, while simultaneously moving one or both of the guidewire 54 with respect to the guidewire driver 64 and the catheter 52 with respect to the catheter driver 62 can also accomplish this motion of the guidewire 54 with respect to the catheter 52.

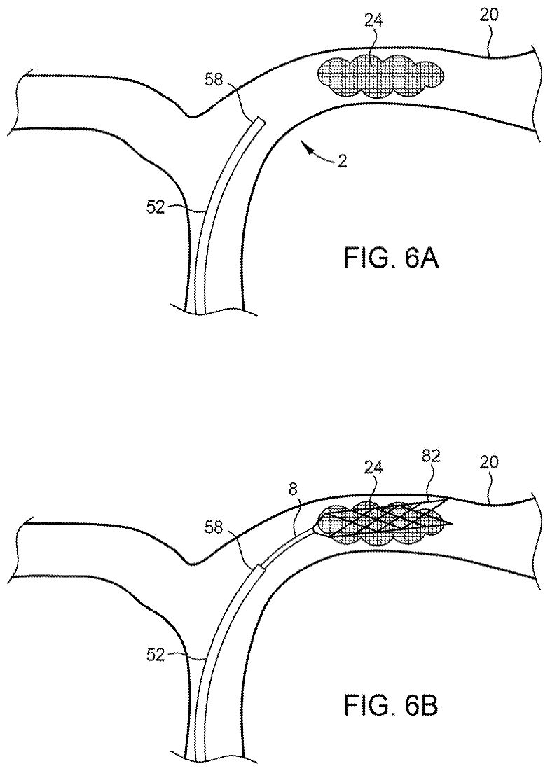

The catheter driver 62 includes two pairs of pinch roller assemblies 68a, 68b (FIGS. 5). Each of the pairs of pinch roller assemblies 68a, 68b includes two rollers 70a, 70b, rotatably supported on drive shafts 72a, 72b extending generally perpendicularly to the drive path of the catheter 52 therethrough. Because roller 70b itself, or pinch roller assembly 68a, is physically driven such as by a motor (not shown), the pinching of the catheter between rollers 70a, 70b of pinch roller assembly 68b, where the rotation of roller 70b is physically coupled to roller 70a through the catheter 52 pinched therebetween to cause the roller 70a to rotate, corresponding linear movement of the catheter 52 captured therebetween occurs. Because the rollers 70a, 70b function as pinch rollers pinching the catheter 52 outer surface therebetween, only one of the two 70a, 70b of each pair 68a, 68b need be driven, and the other roller provides a follower roller surface. The guidewire driver 64 is moveable linearly with respect to the catheter driver 62, and it also includes the same roller construct as that of the catheter driver 62. Because roller 70b itself, or pinch roller assembly 68a is physically driven, the pinching of the guidewire 54 between rollers 70a, 70b of pinch roller assembly 68b causes corresponding linear movement of the guidewire 54 captured therebetween, and the motion of the guidewire 54 causes the rotation of the roller 70b to cause corresponding rotation of roller 70a in the opposite rotational direction. Again, as the rollers 70a, 70b function as pinch rollers pinching the guidewire 54 outer surface therebetween, only one of the two 70a, 70b of each pair 68a, 68b of rollers 70a, 70b need be driven, and the other roller provides a follower roller surface. Alternatively, a second pair of bevel gears connected to a second output shaft of the motor (not shown) driving the roller 70b, or a second motor and bevel gear set, can be provided to drive roller 70a of pinch roller assembly 68a.

To properly position the catheter 52 distal end 58 in a patient lumen, the catheter 52, with the guidewire 54 extending therealong and therethrough, is initially introduced into a patient incision, and advanced along a patient body lumen, for example a blood vessel, while being radiologically imaged for viewing by a surgeon. This may initially be done manually, thereafter, the surgeon, while viewing the lumen, and the controllably bendable distal portion 56 of the guidewire 54 and the location of the catheter distal end 58 radiologically, actuates a joystick or other device to control advancement, retraction, and rotation of the guidewire 54 and the catheter concurrently or independently, as well as the bending orientation of the distal portion 56 of the guidewire 54. As a result, the surgeon is able to direct the guidewire 54 within the patient, and thus to advance the catheter 52 distal end 58, to a desired location within a patient's body or body lumen.

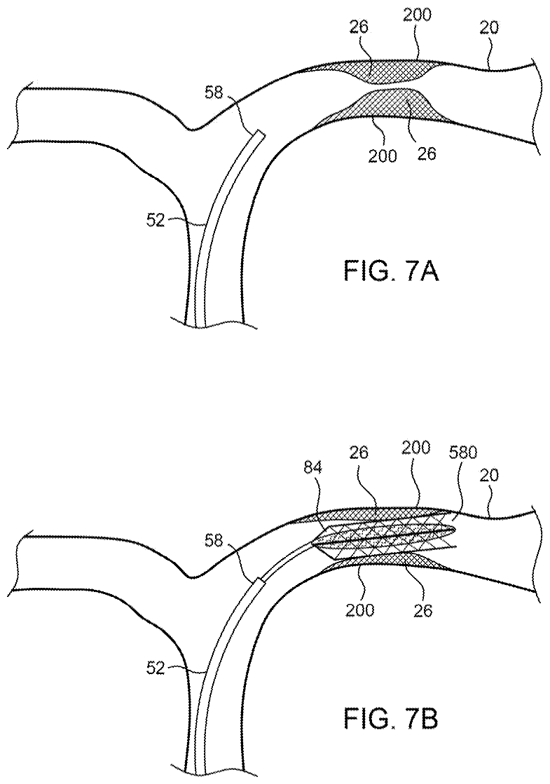

Once the distal end 58 of the catheter 52 is properly positioned in the patient's body or body lumen, a deployable element may be further delivered or retracted through within one or more working lumens of the catheter to the desired location. FIGS. 6A to B are schematic views illustrating a deployable element deployed through the above-mentioned steerable guidewire/catheter system for treating ischemic stroke. The deployable element 8 here is a thrombectomy device such as a stent or stent retriever 82 for treating ischemic stroke. In the example shown in FIGS. 6A and 6B, the deployable device is a stent retriever 82. To deploy the stent retriever 82, the catheter 52 is advanced through the vasculature 2 (FIG. 1), including into blood vessel 20, to reach an occlusion 24 within a patient's blood vessel 20 (FIG. 6A). Once the distal end 58 of the catheter 52 is properly positioned with respect to the occlusion 24, a stent or stent retriever 82 is further deployed from an interior lumen (not shown) of the catheter 52 to the distal end 58 of the catheter 52 and then expanded outwardly to form a collapsible sleeve, net or basket structure to at least partially engage the occlusion 24. Next, the occlusion 24 is aspirated (pulled) toward the distal end 58 of the lumen of the catheter via an aspiration pump (not shown) connected to the proximal end of the catheter (not shown) to cause a suction effect at the distal end 58 of the catheter 52, so that the occlusion 24 is captured within the stent/retriever 82. Thereafter, the occlusion 24 is removed from the blood vessel 20 by the retraction of the stent retriever 82 from the blood vessel.

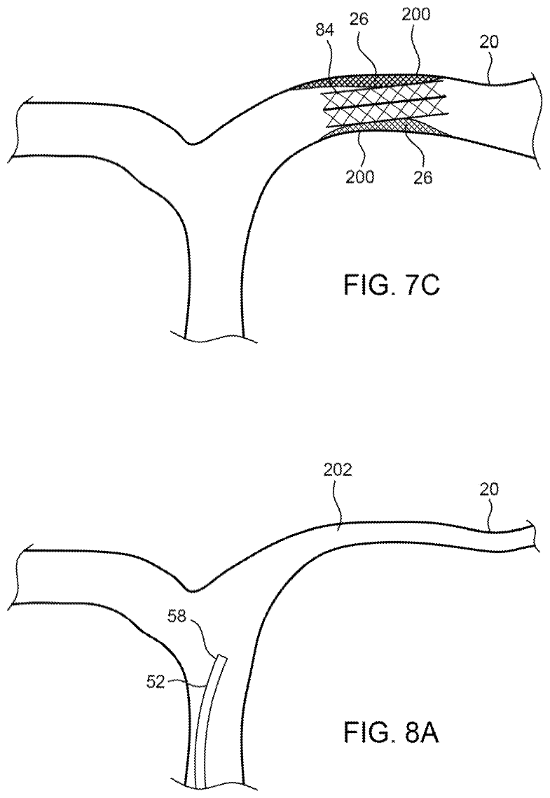

FIGS. 7A to C are schematic views illustrating another deployable element being deployed through the above-mentioned steerable guidewire/catheter system to perform angioplasty. Here, a balloon catheter 52 is provided, including an inflatable balloon 580 at the catheter distal end 58. The catheter 52 is advanced through the vasculature 2 to reach plaques 26 on the walls 200 of the blood vessel 20, (FIG. 7A). Once the catheter distal end 58 is properly positioned adjacent to the plaques, the balloon is inflated to flatten or compress the plaque 26 against the walls 200 (FIG. 7B), so that the narrowed vessel 20 can be opened. In other examples, the inflatable balloon 580 is covered with a stent 84, which is an expandable mesh-like tubular device commonly made of metal. With the inflation of the balloon 580, the stent 84 can be also expanded radially outwardly from a collapsed state thereof toward the walls of the blood vessel 20 to flatten or compress the plaque 26 and provide a tubular pathway through the plaque 26 region of the blood vessel 20 (FIG. 7B). When the balloon 580 is deflated and retracted into the catheter, the stent 84 is left within the blood vessel 20 (FIG. 7C) to keep the vessel 20 open, thereby improving blood flow to the heart muscle and reducing the pain of angina.

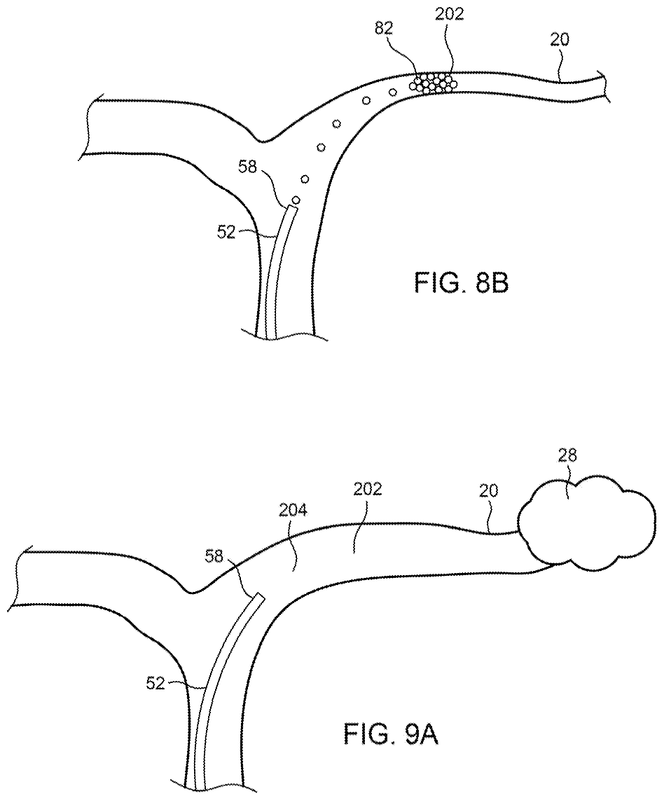

FIGS. 8A to B are schematic views illustrating another deployable element being deployed through the above-mentioned steerable guidewire/catheter system to perform embolization, which is an minimally invasive surgical technique to prevent blood flow to an area of the body so that it can effectively shrink a tumor or block an aneurysm. The deployable element 8 here includes a plurality of degradable microspheres 86 such as tiny gelatin sponges or beads. The catheter 52 is advanced through the vasculature 2 to reach a location at or adjacent to a desired portion 202 of the blood vessel 20 supplying blood flow to a tumor or abnormal area of tissue (FIG. 8A) When the catheter distal end 58 is properly positioned adjacent to the desired location of the deployment of the degradable microspheres 86 , as shown in FIG. 8B,the degradable microspheres 86 are deployed from a lumen (no shown) of the catheter 52 to the catheter distal end 58 and then outwardly of the distal end 58 of the catheter 52 to accumulate together to block or extend over and across the portion 202 of the vessel 20, thereby stopping bleeding or the flow of blood to the tumor or abnormal area downstream, in a blood flow direction of blood vessel 20, therefrom.

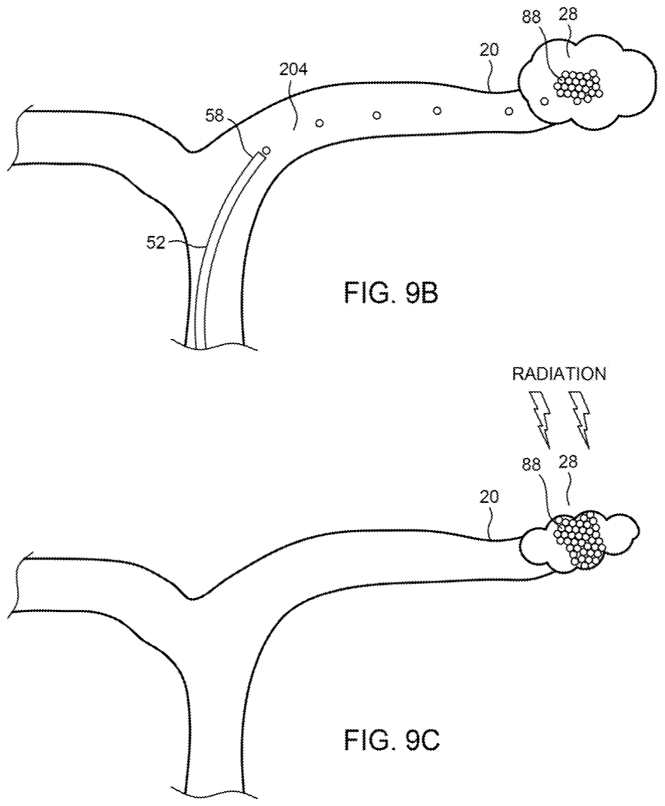

FIGS. 9A to C are schematic views illustrating another deployable element being deployed through the above-mentioned steerable guidewire/catheter system to perform radiation therapy. The deployable element 8 here includes a plurality of radioactive seeds 88 such as tiny gelatin sponges or beads. The catheter 52 is advanced through the vasculature 2 to reach a location at or adjacent to a desired portion 202 of the blood vessel 20, itself adjacent to a tumor area 28 (FIG. 9A) When the catheter distal end 58 is properly positioned at or adjacent to the desired portion 202, as shown in FIG. 9B, the radioactive seeds 88 are deployed from a lumen (not shown) of the catheter 52 to the catheter distal end 58 and thence into or around the tumor area 28, thus allowing doses of radiation to be given off to the tumor area 28 to kill the tumor cells while sparing the surrounding healthy tissue (FIG. 9C).

It is to be noted that various modifications or alterations can be made to the above-described exemplary embodiments of the invention without departing from the technical features of the invention as defined in the appended claims.

What is claimed is:

1. A method for deploying a medical device to a target site of a patient, comprising:
a) introducing a steerable guidewire to a body lumen of a patient, wherein the steerable guidewire having a bendable distal portion comprising an ionic electroactive polymer actuator;
b) steering the bendable distal portion of the guidewire to a target site of the body lumen with a guidewire controller;
c) introducing and advancing a flexible, elongated catheter having a distal end over the guidewire to position the distal end in the target site; and
d) deploying a deployable element through the catheter to the target site.

2. The method of claim 1, wherein the guidewire of b) is removed when the bendable distal portion is positioned.

3. The method of claim 1, wherein the guidewire controller of b) is a hand-held controller.

4. The method of claim 1, where the movement of the guidewire and catheter are controlled by a robotic controller.

5. The method of claim 4, wherein the robotic controller comprises:
a catheter driver configured to advance and retract a catheter having a hollow interior along a catheter advance and retract path extending therein;
a guidewire driver configured to advance and retract a guidewire along a guidewire advance and retract path extending therein; wherein
each of the catheter advance and retract path and the guidewire advance and retract path extend between pairs of rollers in the respective catheter and roller drivers, and the paths are parallel to each another.

6. The method of claim 1, wherein the deployable element is a thrombectomy device.

7. The method of claim 6, the thrombectomy device comprises a mechanical retriever or aspiration assembly.

8. The method of claim 1, wherein the deployable element is a detachable coil.

9. The method of claim 1, wherein the deployable element is a balloon or a stent.

10. The method of claim 1, wherein the deployable element is a therapeutic agent.

11. The method of claim 10, the therapeutic agent is a plurality of microsphere beads or radioactive seeds.
